(19) United States
(12) Patent Application Publication
(10) Pub. No.: US 2007/0281875 A1
(43) Pub. Date: Dec. 6, 2007

(54) Process for decarboxylation of fatty acids and oils to produce paraffins or olefins

(76) Inventors: Jeffrey John Scheibel, Loveland, OH (US); Randall Thomas Reilman, Cincinnati, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/804,849

(22) Filed: May 21, 2007

Related U.S. Application Data
(60) Provisional application No. 60/802,054, filed on May 19, 2006.

(51) Int. Cl. C11D 3/50; C11D 9/44; C07C 2/00; C07C 1/00

(52) U.S. Cl. 510/101; 585/324; 585/638

(57) ABSTRACT

The invention is directed to a process of decarboxylation of natural fats and oils comprising the use of an activated acidic catalyst essentially free of Group VIII metals. Embodiments of the present process comprise the steps of supplying a feedstock comprising of natural fats, oils and mixtures thereof to a reaction vessel, subjecting the feedstock to an activated acidic catalyst essentially free of Group VIII metals, purging the reaction vessel with an inert gas; and subjecting the feedstock and catalyst to a temperature of from about 250° C. to about 500° C.

### CROSS REFERENCE TO RELATED APPLICATIONS

This application claims benefit under 35 U.S.C. §119(e) to U.S. Application Ser. No. 60/802,054 filed May 19, 2006.

### FIELD OF THE INVENTION

The present invention is directed to the production of paraffin and/or olefin compounds from natural vegetable oils and animal fats.

### BACKGROUND OF THE INVENTION

Conversion of vegetable oils and animal fats into paraffinic and olefinic compounds is widely studied and becoming more important as crude oil prices rise and environmental concerns continue to become more important. Paraffins can be used directly as fuels or fuel additives. Both paraffins and olefins are also used as feedstocks for the production of surfactants needed in other consumer product industries. A natural source of surfactants from natural fats and oils via paraffins and olefins continues to be critical to the consumer product manufacturers.

### SUMMARY OF THE INVENTION

Accordingly, it is an object of the present invention to provide an improved process for producing paraffins and olefins from natural fats and oils. Generally, the invention is directed to a process of decarboxylation of natural fats and oils comprising the use of an activated acidic catalyst essentially free of Group VIII metals. Embodiments of the present process comprise the steps of supplying a feedstock comprising of natural fats, oils and mixtures thereof to a reaction vessel, subjecting the feedstock to an activated acidic catalyst essentially free of Group VIII metals, purging the reaction vessel with an inert gas; and subjecting the feedstock and catalyst to a temperature of from about 250° C. to about 500° C.

### DETAILED DESCRIPTION OF THE INVENTION

The present invention relates to a process for decarboxylation of natural fats and oils to produce paraffins and olefins.

Natural fats and oils used in the process of the present invention may comprise fatty acids, alkyl esters of fatty acids, and mixtures thereof. Embodiments of the natural fats of the present invention are. Examples of such compositions include, but are not limited to C₈₋₂₂ fatty acids, mono- and di-glycerides of C₈₋₂₂ fatty acids, C₁₋₄ alkyl esters of C₈₋₂₂ fatty acids, triglycerides with C₈₋₂₂ fatty acid, palm oil, coconut oil, tallow fat, and mixtures thereof.

These fats and oils are converted to paraffins and olefins by decarboxylation or deoxygenation of the oxygen containing carbonyl or ester group on the compound. Critical to the process of the present invention is the tightly controlled selection of an activated acidic catalyst. The catalyst may be selected from shape-selective moderately acidic catalysts. One embodiment of the shape-selective moderately acidic catalyst is zeolite, preferably selected from the group consisting of mordenite, ZSM-4, ZSM-12, ZSM-20, offretite, gmelinite and zeolite beta in at least partially acidic form. In another embodiment the zeolite is substantially in acid form and is contained in a catalyst pellet comprising a conventional binder and further wherein said catalyst pellet comprises at least about 1%, more preferably at least 5%, more typically from 50% to about 90%, of said zeolite. More generally, suitable catalysts may be at least partially crystalline not including binders or other materials used to form catalyst pellets.

The pores characterizing the zeolites useful in the present invention may be substantially circular, such as in cancrinite which has uniform pores of about 6.2 angstroms, or preferably may be somewhat elliptical, such as in mordenite. In any case, the zeolites have a major pore dimension intermediate between that of the large pore size dimensions, such as the X and Y zeolites, and the relatively small pore size zeolites ZSM-5 and ZSM-11, and are preferably between about 6 angstroms and about 7 angstroms.

The zeolites useful in the decarboxylation of the present invention may have at least 10 percent of the cationic sites thereof occupied by ions other than alkali or alkaline-earth metals. Typical, but non-limiting replacing ions include ammonium, hydrogen, rare earth, zinc, copper and aluminum. In some specific embodiments, ammonium, hydrogen, rare earth elements or combinations thereof are used as the replacing ions. In certain embodiments, the extent of replacement is such as to produce a zeolite material in which at least 50 percent of the cationic sites are occupied by hydrogen ions.

The zeolites may be subjected to various chemical treatments, including alumina extraction (dealumination) and combination with Group IIB, III, IV, VI, VII metals. Certain embodiments include zeolites in combination with the Group VI-B metals, tungsten, molybdenum, and chromium, and oxides of the Group VI-B metals. It is also contemplated that the zeolites may be subjected to thermal treatment, including steaming or calcination in air, hydrogen or an inert gas.

In addition to the foregoing materials, the acidic catalysts of the present invention may be compounded with a porous matrix material, such as alumina, silica-alumina, silica-magnesia, silica-zirconia, silica-thoria, silica-beryllia, and silica-alumina-zirconia, as well as ternary combinations of these materials. A group of zeolites which may be used in the process of the present invention have a silica:alumina ratio of at least 10:1, preferably at least 20:1. The silica:alumina ratios referred to are the structural or framework ratios, that is the ratio for the SiO₄ to the AlO₄ tetrahedra.

Zeolite beta suitable for use herein is disclosed in U.S. Pat. No. 3,308,069. Such a zeolite beta in the acid form is commercially available as Zeocat PB/H® from Zeochem. Acidic mordenite type catalysts also suitable for use herein is described in European Patent Application EP 0 466 558. Another acidic mordenite catalyst useful for the decarboxylation process of the present invention is disclosed in U.S. Pat. No. 4,861,935 which relates to a hydrogen form mordenite incorporated with alumina where the composition has a surface area of at least 580 m²/g. Other acidic mordenite catalysts useful herein include those disclosed in U.S. Pat. No. 5,243,116, U.S. Pat. No. 5,198,595, and U.S. Pat. No. 5,175,135.

As with many reactions, the rate of the decarboxylation reaction is impacted by the temperature and pressure at which reaction occurs. Generally, the decarboxylation process of the present invention is performed at a temperature ranging from about 250° C. to about 500° C. Certain embodiments of the process are performed at from about 275° C. to about 325° C. The process is performed under inert atmosphere at a positive pressure ranging from about 0.1 psig to about 4000 psig. Selected embodiments of the process may be performed at from about 10 psig to about 1000 psig, with some embodiments having a pressure of from about 100 psig to about 500 psig. The positive inert atmosphere may be achieved by purging and charging the vessel to pressure with any non-oxidizing gas, such as nitrogen, argon, hydrogen or methane.

The decarboxylation process of the present invention may be performed via a batch process, a semi-continuous process or a continuous process. Embodiments utilizing a batch process may use any standard pressure vessel capable of maintaining the required temperatures and pressures with mechanical or magnetic agitation. Embodiments of batch processes include processes where the natural fats and/or oils are charged into the vessel with the selected acidic catalyst, heated to a temperature of from about 275° C. to about 325° C. under positive pressure for a time period of 5 minutes to 180 minutes depending on the catalyst. Embodiments of a continuous process involve processes where the fat and/or oil is passed through a continuous reaction vessel over a bed consisting of the acidic catalyst at a temperature of from about 300° C. to about 500° C. under inert positive pressure for a contact time of from about 0.1 seconds to about 900 seconds (15 minutes).

The desired form, paraffin or olefin, of the products of the decarboxylation process of the present invention may be obtained by specific selection of the catalyst and or adding additional certain reaction solvents into the reaction vessel. Higher levels of saturated paraffinic material may be obtained by including gases with higher levels of available hydrogen in the pressurizing gas. For example, adding hydrogen gas to one of the other inert gases will yield more saturated compounds. Also, it has been found that some of the acidic catalysts have available hydrogen build into the catalyst structure. For example beta zeolites and acidic mordenite catalyst will promote more saturation during the decarboxylation.

Higher levels of unsaturated olefinic material may be obtained by performing the decarboxylation reaction in an environment with little or no available hydrogen. For example, using nitrogen or inert gases to pressurize the vessel will yield more unsaturated compounds. Further, it has been found that acidic zeolites chemically treated with Group VI-B metals will promote less saturation during decarboxylation and produce more olefinic materials.

The actual time (batch) or contact time (continuous) required for the decarboxylation reaction must be carefully controlled based on the selection of the catalyst, process temperature and pressure. The paraffin/olefin mixture must be removed from the reaction vessel and catalyst immediately upon formation to prevent cracking to shorter paraffins/olefins. The desired paraffin and/or olefin products are removed from the reaction mixture by standard distillation processes.

Another way to control the amount of destructive cracking of the reaction material is by adding a small amount of Group VIII metal, such as nickel, palladium and platinum, Group VIII metal/carbon structures or Group VIII metal oxides to the activated acidic catalyst of the present invention. The Group VIII catalysts are generally undesirable in the process of the present invention, but where cracking may be a risk less than 10% of the Group VIII catalyst may be added to make a multi-catalyst system. Other embodiment include catalyst systems where the Group VIII catalyst comprises from about 0.001% to about 5% and where the Group VIII catalyst comprises from about 0.1% to 2% of the catalyst system. Additionally, when a Group VIII catalyst is used in the process of the present invention to prevent cracking, a low level of hydrogen may be incorporated into the non-oxidating pressurization gas. When needed the hydrogen may be used and less than 5%, less then 3% or even less than 1% of the gas.

The process of the present invention will generally have a linear/branching distribution similar to the distribution of the incoming feedstock. However, it is possible that a small amount of branching may occur during the decarboxylation process of the present invention. Higher levels of branching may be desirable or undesirable depending on the proposed us of the final paraffin/olefin product. If the objective is to produce paraffins for fuels or fuel additives higher branching is not desirable. However, if the paraffins and olefins are to be converted into cleaning surfactants higher levels of branching may, in fact, be beneficial. If desired, even higher branching levels may be achieved by operating the process at higher temperatures or for longer times which taking steps to prevent cracking.

It may be desirable to produce olefins in a two step process where the fats and oils are converted to paraffins first using the decarboxylation process of the present invention and then converting the paraffins to olefins via dehydrogenation. Dehydrogenation of the paraffin or olefin/paraffin mixtures in the instant process can be accomplished using any of the well-known dehydrogenation catalyst systems, including those described in the Surfactant Science Series references cited in the background as well as in “Detergent Manufacture Including Zeolite Builders and Other New Materials”, Ed. Sittig, Noyes Data Corp., New Jersey, 1979 and other dehydrogenation catalyst systems, for example those commercially available though UOP Corp. Dehydrogenation can be conducted in presence of hydrogen gas and commonly a precious metal catalyst (e.g., DeH-5®, DeH-7®, DeH-9® available from UOP) is present though alternatively non-hydrogen, precious-metal free dehydrogenation systems such as a zeolite/air system can be used with no precious metals present.

More specifically, dehydrogenation catalysts useful herein include a catalyst supported on Sn-containing alumina and having Pt: 0.16%, Ir: 0.24%, Sn: 0.50%, and Li: 0.54% as described in U.S. Pat. No. 5,012,027 incorporated by reference. This catalyst, when contacted with a C9-C14 paraffin mixture (believed to be linear) at 500° C. and 0.68 atm. produces olefinic products (38 h on stream) with 90.88% selectivity and 11.02% conversion and is believed to be very suitable for at least partially dehydrogenating branched-enriched streams of paraffins herein. See also U.S. Pat. No. 4,786,625; EP 320,549 A1 Jun. 21, 1989; Vora et al., Chem. Age India (1986), 37(6), 415-18;

Other useful dehydrogenation systems readily adapted into the present invention include those of U.S. Pat. No. 4,762,960 incorporated by reference which discloses a Pt-group metal containing dehydrogenation catalyst having a modifier metal selected from the group consisting of Sn, Ge, Re and their mixtures, an alkali metal, an alkaline earth metal or their mixtures, and a particularly defined refractory oxide support. Alternative dehydrogenation catalysts and conditions useful herein include those of U.S. Pat. No. 4,886,926 and of U.S. Pat. No. 5,536,695.

As briefly discussed above, the paraffins and olefins produced via the process of the present invention may be used as paraffins and olefins are used today. These uses include fuels, fuel additives and as a starting material for many industrial products including cleaning surfactants.

### Cleaning Surfactants

Cleaning surfactants as used herein, include without limitation such surfactants as paraffin sulfonate, linear alkyl benzenes, alkyl sulfates, alkyl sulfonates, and ethoxylated alkyl sulfonates. The cleaning surfactants are be produced using the paraffins and/or olefins of the present invention disclosed above by functionalizing and/or derivatizing the hydrocarbon by using processes known in the industry.

The olefin and/or paraffin is functionalized by adding an aromatic group to the hydrocarbon via alkylation or by converting the hydrocarbon to an alcohol by typical OXO processes.

### Alkylation

Alkylation is conducted by reacting the paraffin/olefin hydrocarbon with an aromatic hydrocarbon selected from benzene, toluene and mixtures thereof in the presence of an alkylation catalyst. Numerous alkylation catalysts are readily available for use. Alkylation catalysts include the DETAL® process catalysts, aluminum chloride, HF, HF on zeolites, fluoridated zeolites, non-acidic calcium mordenite, shape-selective moderately acidic alkylation catalysts, preferably zeolitic. The zeolite in such catalysts for the alkylation step is preferably selected from the group consisting of mordenite, ZSM-4, ZSM-12, ZSM-20, offretite, gmelinite and zeolite beta in at least partially acidic form. More preferably, the zeolite is substantially in acid form and is contained in a catalyst pellet comprising a conventional binder and further wherein said catalyst pellet comprises at least about 1%, more preferably at least 5%, more typically from 50% to about 90%, of said zeolite.

More generally, suitable alkylation catalyst is typically at least partially crystalline, more preferably substantially crystalline not including binders or other materials used to form catalyst pellets, aggregates or composites. Moreover the catalyst is typically at least partially acidic. H-form mordenite is preferable.

Zeolite beta suitable for use herein (but less preferred than H-mordenite) is disclosed in U.S. Pat. No. 3,308,069. Such a zeolite in the acid form is also commercially available as Zeocat PB/H from Zeochem.

EP 466,558 describes an acidic mordenite type alkylation catalyst also of possible use herein having overall Si/Al atomic ratio of 15-85 (15-60), Na weight content of less than 1000 ppm (preferably less than 250 ppm), having low or zero content of extra-network Al species, and an elementary mesh volume below 2,760 nm3. U.S. Pat. No. 5,057,472 useful for preparing alkylation catalysts herein relates to concurrent dealumination and ion-exchange of an acid-stable Na ion-containing zeolite, preferably mordenite effected by contact with a 0.5-3 (preferably 1-2.5) M HNO3 solution containing sufficient NH4NO3 to fully exchange the Na ions for NH4 and H ions. The resulting zeolites can have an SiO2:Al2O3 ratio of 15-26 (preferably 17-23):1 and are preferably calcined to at least partially convert the NH4/H form to an H form. Optionally, though not necessarily particularly desirable in the present invention, the catalyst can contain a Group VIII metal (and optionally also an inorganic oxide) together with the calcined zeolite of '472.

Another acidic mordenite catalyst useful for the alkylation step herein is disclosed in U.S. Pat. No. 4,861,935 which relates to a hydrogen form mordenite incorporated with alumina, the composition having a surface area of at least 580 m2/g. Other acidic mordenite catalysts useful for the alkylation step herein include those described in U.S. Pat. No. 5,243,116 and U.S. Pat. No. 5,198,595. Yet another alkylation catalyst useful herein is described in U.S. Pat. No. 5,175,135 which is an acid mordenite zeolite having a silica/alumina molar ratio of at least 50:1, a Symmetry Index of at least 1.0 as determined by X-ray diffraction analysis, and a porosity such that the total pore volume is in the range from about 0.18 cc/g to about 0.45 cc/g and the ratio of the combined meso- and macropore volume to the total pore volume is from about 0.25 to about 0.75. Particularly preferred alkylation catalysts herein include the acidic mordenite catalysts Zeocat™ FM-8/25H available from Zeochem; CBV 90 A available from Zeolyst International, and LZM-8 available from UOP Chemical Catalysts.

### OXO Reaction

The olefins and paraffins of the present invention may also be functionalized by converting hydrocarbons into useful modified primary alcohols which can be used as a cleaning surfactant directly or to make other derivatives such as soluble sulfates, poly(alkoxy)sulfates, and poly(alkoxylates). The modified versions of any other surfactant types known in the art to be derivable from OXO alcohols are, of course, included in the present invention.

U.S. Pat. No. 5,780,694 and WO 98/23566 include a description of one OXO process. Surfactant Science Series, Volume 7, “Anionic Surfactants”, Part 1, Marcel Dekker, N. Y., Ed. W. Linfield, 1976, Chapter 2 “Petroleum-Based Raw Materials for Anionic Surfactants”, pages 11-86 provides general background including for the OXO process. The OXO process discussion therein shows conversion of linear olefin to mixtures of “branched” and linear alcohols. Separately, Kirk Othmer's Encyclopedia of Chemical Technology, 4^{th}. Edition, Vol. 1, pages 893-913 (1991), article entitled “Alcohols, Higher Aliphatic”, sub-heading “Synthetic Processes” describes an OXO reaction to form detergent alcohols. See also WO97/01521 A1 published Jan. 16, 1997 and 95 ZA-0005405 published Jun. 25, 1995. See also various technical bulletins and publications of Sasol and/or Sastech of South Africa, especially in relation to already known or available OXO alcohols made or makable by the OXO processes proprietary to these companies.

### Sulfonation/Sulfation/Alkoxylation and Workup

In general, sulfonation of the modified alkylbenzenes or sulfation of modified primary OXO alcohols (or their alkoxylates) in the instant process can be accomplished using any of the well-known sulfonation systems, including those described in “Detergent Manufacture Including Zeolite Builders and Other New Materials” as well as in the Surfactant Science Series review of alkylbenzenesulfonate manufacture. Common sulfonation systems include sulfuric acid, chlorosulfonic acid, oleum, sulfur trioxide and the like. Sulfur trioxide/air is especially preferred. Details of sulfonation using a suitable air/sulfur trioxide mixture are provided in U.S. Pat. No. 3,427,342. Sulfonation processes are further extensively described in “Sulfonation Technology in the Detergent Industry”, W. H. de Groot, Kluwer Academic Publishers, Boston, 1991.

Any convenient workup steps may be used in the present process. Common practice is to neutralize after sulfonation with any suitable alkali. Thus the neutralization step can be conducted using alkali selected from sodium, potassium, ammonium, magnesium and substituted ammonium alkalis and mixtures thereof. Potassium can assist solubility, magnesium can promote soft water performance and substituted ammonium can be helpful for formulating specialty variations of the instant surfactants. The invention encompasses any of these derivative forms of the modified alkylbenzenesulfonate surfactants, or of the sulfated modified primary OXO alcohols, or of the alkoxylated, sulfated modified primary OXO alcohols as produced by the present process and their use in consumer product compositions.

### Laundry Detergents and Cleaning Compositions

The cleaning surfactants of the present invention can be incorporated into cleaning detergent compositions such as liquid and granular laundry detergents, hand dish detergents, automated dish detergents, hard surface cleaners, solid and liquid soaps, and hair shampoos. The inventive laundry detergents and cleaning compositions of the present invention comprise generally from 0.05 to 35% by weight, preferably from 1 to 20% by weight and more preferably from 0.5 to 20% by weight, based on the particular overall composition, of the cleaning surfactants.

In addition, the laundry detergents and cleaning compositions generally may comprise other surfactants and, if appropriate, polymers as washing substances, builders and further customary ingredients, for example cobuilders, complexing agents, bleaches, standardizers, graying inhibitors, dye transfer inhibitors, enzymes and perfumes.

The cleaning surfactants of the present invention may be utilized in laundry detergents or cleaning compositions in surfactant systems comprising C₁₀-C₁₅ alkyl benzene sulfonates (LAS) made by the present process and one or more co-surfactants selected from nonionic, cationic, anionic or mixtures thereof. The selection of co-surfactant may be dependent upon the desired benefit. In one embodiment, the co-surfactant is selected as a nonionic surfactant, preferably C₁₂-C₁₈ alkyl ethoxylates. In another embodiment, the co-surfactant is selected as an anionic surfactant, preferably C₁₀-C₁₈ alkyl alkoxy sulfates (AEₓS) wherein x is from 1-30. In another embodiment the co-surfactant is selected as a cationic surfactant, preferably dimethyl hydroxyethyl lauryl ammonium chloride. If the detergent composition comprises a cleaning surfactant functionalized to C₁₀-C₁₅ alkyl benzene sulfonates (LAS), the LAS is used at levels ranging from about 9% to about 25%, or from about 13% to about 25%, or from about 15% to about 23% by weight of the composition.

The surfactant system may comprise from 0% to about 7%, or from about 0.1% to about 5%, or from about 1% to about 4% by weight of the composition of a co-surfactant selected from a nonionic co-surfactant, cationic co-surfactant, anionic co-surfactant and any mixture thereof.

Examples of nonionic co-surfactants that may be made by the present process and used in the detergent compositions include: C₁₂-C₁₈ alkyl ethoxylates; C₆-C₁₂ alkyl phenol alkoxylates wherein the alkoxylate units are a mixture of ethyleneoxy and propyleneoxy units; C₁₂-C₁₈ alcohol and C₆-C₁₂ alkyl phenol condensates with ethylene oxide/propylene oxide block alkyl polyamine ethoxylates; C₁₄-C₂₂ mid-chain branched alcohols; C₁₄-C₂₂ mid-chain branched alkyl alkoxylates; and ether capped poly(oxyalkylated) alcohol surfactants as discussed in U.S. Pat. No. 6,482,994 and WO 01/42408.

Non-limiting examples of semi-polar nonionic co-surfactants that may be used in the detergent compositions include: water-soluble amine oxides containing one alkyl moiety of from about 10 to about 18 carbon atoms and 2 moieties selected from the group consisting of alkyl moieties and hydroxyalkyl moieties containing from about 1 to about 3 carbon atoms; water-soluble phosphine oxides containing one alkyl moiety of from about 10 to about 18 carbon atoms and 2 moieties selected from the group consisting of alkyl moieties and hydroxyalkyl moieties containing from about 1 to about 3 carbon atoms; and water-soluble sulfoxides containing one alkyl moiety of from about 10 to about 18 carbon atoms and a moiety selected from the group consisting of alkyl moieties and hydroxyalkyl moieties of from about 1 to about 3 carbon atoms.

Non-limiting examples of cationic co-surfactants that may be used in the present detergent compositions include: quaternary ammonium surfactants which can have up to 26 carbon atoms including alkoxylate quaternary ammonium (AQA) surfactants, dimethyl hydroxyethyl quaternary ammonium; dimethyl hydroxyethyl lauryl ammonium chloride; polyamine cationic surfactants; cationic ester surfactants; and amino surfactants.

Nonlimiting examples of anionic co-surfactants which may be made by the present invention and may be useful herein include C₁₀-C₂₀ primary, branched chain and random alkyl sulfates (AS); C₁₀-C₁₈ secondary (2,3) alkyl sulfates; C₁₀-C₁₈ alkyl alkoxy sulfates (AEₓS) wherein x is from 1-30; C₁₀-C₁₈ alkyl alkoxy carboxylates comprising 1-5 ethoxy units; mid-chain branched alkyl sulfates; mid-chain branched alkyl alkoxy sulfates; modified alkylbenzene sulfonate (MLAS); methyl ester sulfonate (MES); and alpha-olefin sulfonate (AOS).

The present invention relates to detergent and cleaning compositions comprising the cleaning surfactants made from the paraffins and/or olefins produced from fats and oils according to the new decarboxylation process. The compositions can be in any form, namely, in the form of a liquid; a solid such as a powder, granules, agglomerate, paste, tablet, pouches, bar, gel; an emulsion; types delivered in dual-compartment containers; a spray or foam detergent; premoistened wipes (i.e., the cleaning composition in combination with a nonwoven material such as that discussed in U.S. Pat. No. 6,121,165, Mackey, et al.); dry wipes (i.e., the cleaning composition in combination with a nonwoven materials, such as that discussed in U.S. Pat. No. 5,980,931, Fowler, et al.) activated with water by a consumer; and other homogeneous or multiphase consumer cleaning product forms.

In one embodiment, the cleaning composition of the present invention is a liquid or solid laundry detergent composition. In another embodiment, the cleaning composition of the present invention is a hard surface cleaning composition, preferably wherein the hard surface cleaning composition impregnates a nonwoven substrate. As used herein “impregnate” means that the hard surface cleaning composition is placed in contact with a nonwoven substrate such that at least a portion of the nonwoven substrate is penetrated by the hard surface cleaning composition, preferably the hard surface cleaning composition saturates the nonwoven substrate. The cleaning composition may also be utilized in car care compositions, for cleaning various surfaces such as hard wood, tile, ceramic, plastic, leather, metal, glass. This cleaning composition could be also designed to be used in a personal care and pet care compositions such as shampoo composition, body wash, liquid or solid soap and other cleaning composition in which surfactant comes into contact with free hardness and in all compositions that require hardness tolerant surfactant system, such as oil drilling compositions.

In another embodiment the cleaning composition is a dish cleaning composition, such as liquid hand dishwashing compositions, solid automatic dishwashing compositions, liquid automatic dishwashing compositions, and tab/unit does forms of automatic dishwashing compositions.

Quite typically, cleaning compositions herein such as laundry detergents, laundry detergent additives, hard surface cleaners, synthetic and soap-based laundry bars, fabric softeners and fabric treatment liquids, solids and treatment articles of all kinds will require several adjuncts, though certain simply formulated products, such as bleach additives, may require only, for example, an oxygen bleaching agent and a surfactant as described herein. A comprehensive list of suitable laundry or cleaning adjunct materials can be found in WO 99/05242.

Common cleaning adjuncts include builders, enzymes, polymers not discussed above, bleaches, bleach activators, catalytic materials and the like excluding any materials already defined hereinabove. Other cleaning adjuncts herein can include suds boosters, suds suppressors (antifoams) and the like, diverse active ingredients or specialized materials such as dispersant polymers (e.g., from BASF Corp. or Rohm & Haas) other than those described above, color speckles, silvercare, anti-tarnish and/or anti-corrosion agents, dyes, fillers, germicides, alkalinity sources, hydrotropes, anti-oxidants, enzyme stabilizing agents, pro-perfumes, perfumes, solubilizing agents, carriers, processing aids, pigments, and, for liquid formulations, solvents, chelating agents, dye transfer inhibiting agents, dispersants, brighteners, suds suppressors, dyes, structure elasticizing agents, fabric softeners, anti-abrasion agents, hydrotropes, processing aids, and other fabric care agents, surface and skin care agents. Suitable examples of such other cleaning adjuncts and levels of use are found in U.S. Pat. Nos. 5,576,282, 6,306,812 B1 and 6,326,348 B1.

### Method of Use

The present invention includes a method for cleaning a targeted surface. As used herein “targeted surface” may include such surfaces such as fabric, dishes, glasses, and other cooking surfaces, hard surfaces, hair or skin. As used herein “hard surface” includes hard surfaces being found in a typical home such as hard wood, tile, ceramic, plastic, leather, metal, glass. Such method includes the steps of contacting the composition comprising the modified polyol compound, in neat form or diluted in wash liquor, with at least a portion of a targeted surface then optionally rinsing the targeted surface. Preferably the targeted surface is subjected to a washing step prior to the aforementioned optional rinsing step. For purposes of the present invention, washing includes, but is not limited to, scrubbing, wiping and mechanical agitation.

As will be appreciated by one skilled in the art, the cleaning compositions of the present invention are ideally suited for use in home care (hard surface cleaning compositions), personal care and/or laundry applications.

The composition solution pH is chosen to be the most complimentary to a target surface to be cleaned spanning broad range of pH, from about 5 to about 11. For personal care such as skin and hair cleaning pH of such composition preferably has a pH from about 5 to about 8 for laundry cleaning compositions pH of from about 8 to about 10. The compositions are preferably employed at concentrations of from about 200 ppm to about 10,000 ppm in solution. The water temperatures preferably range from about 5° C. to about 100° C.

For use in laundry cleaning compositions, the compositions are preferably employed at concentrations from about 200 ppm to about 10000 ppm in solution (or wash liquor). The water temperatures preferably range from about 5° C. to about 60° C. The water to fabric ratio is preferably from about 1:1 to about 20:1.

The method may include the step of contacting a nonwoven substrate impregnated with an embodiment of the composition of the present invention As used herein “nonwoven substrate” can comprise any conventionally fashioned nonwoven sheet or web having suitable basis weight, caliper (thickness), absorbency and strength characteristics. Examples of suitable commercially available nonwoven substrates include those marketed under the tradename SONTARA® by DuPont and POLYWEB® by James River Corp.

As will be appreciated by one skilled in the art, the cleaning compositions of the present invention are ideally suited for use in liquid dish cleaning compositions. The method for using a liquid dish composition of the present invention comprises the steps of contacting soiled dishes with an effective amount, typically from about 0.5 ml. to about 20 ml. (per 25 dishes being treated) of the liquid dish cleaning composition of the present invention diluted in water.

### EXAMPLES

The following examples illustrate the compositions of the present invention but are not necessarily meant to limit or otherwise define the scope of the invention herein.

### Example 1

Paraffins may be produced via the following process. A beta zeolite catalyst is charged into a standard batch pressure vessel having agitation. The beta zeolite has been or is activated by drying at about 150° C. to 400° C. at 5 mm Hg pressure. A fatty acid or ester, for example stearic acid, is added to the reaction vessel and the vessel is sealed. A positive pressure of about 300 psig of an inert nitrogen or nitrogen-hydrogen mixture is applied to the vessel and the reaction mixture is heated to a temperature of about 300° C. The reaction is allowed to continue under agitation for between 1 and 2 hours. The reaction mixture is then removed from the vessel and distilled to recover the desired paraffin compound, n-heptadecane if stearic acid was used.

### Example 2

Paraffins may be produced via the following process. A bed of beta zeolite catalyst is charged into a standard continuous reaction pressure vessel. The beta zeolite has been or is activated by drying at about 150° C. to 400° C. at 5 mm Hg pressure. A positive pressure of about 300 psig of an inert nitrogen or nitrogen-hydrogen mixture is applied to the vessel and the vessel and catalyst bed are heated to a temperature of about 400° C. A fatty acid or ester, for example ethyl stearate, is passed over the catalyst bed with a contact time of between 2 and 5 minutes. The reaction mixture is collected and distilled to recover the desired paraffin compound, n-heptadecane if ethyl stearate was used.

### Example 3

Olefins may be produced via the following process. A zeolite catalyst chemically treated with tungsten oxide is charged into a standard batch pressure vessel having agitation. The zeolite-tungsten oxide catalyst has been or is activated by drying at about 150° C. to 400° C. at 5 mm Hg pressure. A fatty acid or ester, for example stearic acid, is added to the reaction vessel and the vessel is sealed. A positive pressure of about 300 psig of an inert nitrogen or argon is applied to the vessel and the reaction mixture is heated to a temperature of about 300° C. The reaction is allowed to continue under agitation for between 1 and 2 hours. The reaction mixture is then removed from the vessel and distilled to recover the desired olefin compound, heptadecene if stearic acid was used.

### Example 4

Olefins may be produced via the following process. A bed of zeolite catalyst chemically treated with tungsten oxide is charged into a standard continuous reaction pressure vessel. The zeolite-tungsten oxide catalyst has been or is activated by drying at about 150° C. to 400° C. at 5 mm Hg pressure. A positive pressure of about 300 psig of an inert nitrogen or argon is applied to the vessel and the vessel and catalyst bed are heated to a temperature of about 400° C. A fatty acid or ester, for example ethyl stearate, is passed over the catalyst bed with a contact time of between 2 and 5 minutes. The reaction mixture is collected and distilled to recover the desired olefin compound, heptadecene if ethyl stearate was used.

### Example 5

Olefins may also be produced via the following process. Paraffins are produced according to the process of Examples 1 or 2. The resulting paraffins are passed continuously to a standard commercial LAB process dehydrogenation unit provided by UOP Corp. (PACOL® process) charged with a nonproprietary Platinum dehydrogenation catalyst.

### CLEANING SURFACTANT EXAMPLES

As noted above, the present invention also encompassed processes of producing cleaning surfactants. The examples that follow are cleaning surfactants produced from the paraffins and/or olefins of the present invention.

### Example 6

If paraffins, such as those from Examples 1 or 2 are used, the paraffins are to a standard commercial LAB process dehydrogenation unit provided by UOP Corp. (PACOL® process) charged with a standard LAB dehydrogenation catalyst (DeH 5® or DeH 7® or similar) proprietary to UOP Corp. The hydrocarbons from either the dehydration step or olefins from Examples 3 through 5 are passed to an alkylation unit which is otherwise conventional but is charged with H-mordenite (ZEOCAT® FM 8/25H) where alkylation proceeds continuously at a temperature of about 200° C. with discharge on reaching a completion of at least about 90%, that is, a conversion of the input hydrocarbon (olefins) of at least about 90%. This produces a modified alkylbenzene. In optional variations, the above procedure can be repeated except with discharge on reaching a conversion (based on olefin) to the desired modified alkylbenzene of at least about 80%. A recycle of any residual paraffins is obtained by distillation at the back-end of the alkylation unit and the recycle is passed back to the dehydrogenation process. The modified alkylbenzene can optionally be further purified by additional conventional distillation. The distilled modified alkylbenzene mixture is sulfonated batchwise or continuously using sulfur trioxide as sulfonating agent. Details of sulfonation using a suitable air/sulfur trioxide mixture are provided in U.S. Pat. No. 3,427,342. The modified alkylbenzenesulfonic acid product of the preceding step is neutralized with sodium hydroxide to give modified alkylbenzene sulfonate, sodium salt mixture.

### Example 7

If paraffins, such as those from Examples 1 or 2 are used, the paraffins are to a standard commercial LAB process dehydrogenation unit provided by UOP Corp. (PACOL® process) charged with a standard LAB dehydrogenation catalyst such as DeH 7® proprietary to UOP Corp. The hydrocarbons from either the dehydration step or olefins from Examples 3 through 5 are passed continuously to an alkylation unit which is otherwise conventional but is charged with H-ZSM 12 where alkylation proceeds continuously at a temperature of about 200° C. with discharge on reaching a conversion of the input hydrocarbon of at least about 90%. The modified alkylbenzene mixture produced in the preceding step is distilled and sulfonated batchwise or continuously using sulfur trioxide as sulfonating agent. The modified alkylbenzenesulfonic acid product of the preceding step is neutralized with sodium hydroxide to give modified alkylbenzene sulfonate, sodium salt mixture.

### Example 8

If paraffins, such as those from Examples 1 or 2 are used, the paraffins are to a standard to a standard commercial LAB process dehydrogenation unit provided by UOP Corp. (PACOL® process) charged with a standard dehydrogenation catalyst (DeH 5® or DeH 7® or similar) proprietary to UOP Corp. The hydrocarbons from either the dehydration step or olefins from Examples 3 through 5 are passed to DEFINE® and PEP® process units licensed from UOP Corp. These units hydrogenate diolefin impurity to monoolefin and help reduce the content of aromatic impurities, respectively. The resulting purified olefin/paraffin stream now passes to an OLEX® process unit licensed from UOP, charged with olefin separation sorbent proprietary to UOP Corp. After olefin separation from unreacted paraffins (the latter are recycled as stream 8 in FIG. 10), the olefinic hydrocarbons are passed continuously to an OXO reaction unit operating with a 2-2.5:1 H2:CO ratio and using a pressure of from about 60-90 atm. and a temperature of about 170° C.− about 210° C. and charged with a cobalt organophosphine complex. OXO proceeds continuously with discharge on reaching a selectivity to the modified primary OXO alcohol of at least about 90%, and essentially all the olefin of the input stream has reacted. This produces a modified primary OXO alcohol according to the invention. A small amount of reduction also occurs to form paraffin. The paraffins are separated by distillation and can be recycled to the dehydrogenation process. The modified primary OXO alcohol is ethoxylated to an average of one mole of ethylene oxide content. Alternatively ethoxylation, propoxylation etc. can be done using differing amounts of alkylene oxide to produce the desired alkoxylate. This is done batchwise or continuously, using ethylene oxide and a base catalyst (see Schonfeldt, Surface Active Ethylene Oxide Adducts, Pergamon Press, N.Y., 1969). The ethoxylated modified OXO alcohol is treated batchwise or continuously with sulfur trioxide as sulfating agent (See “Sulphonation Technology in the Detergent Industry”, W. de Groot, Kluwer Academic Publishers, London, 1991). The product of the preceding step is neutralized with sodium hydroxide to give modified alkyl ethoxysulfate, sodium salt, according to the invention. In variations of the above example, alkyl chain length of the hydrocarbon can be varied so as to produce the desired chainlength modified OXO alcohol derived surfactants as used in the formulation Examples. In a further variation, the modified OXO alcohol can be sulfated without any prior alkoxylation.

### Composition Formulations

As noted above, the present invention further considers detergent compositions made with the cleaning surfactants produced from fats and oils via the decarboxylation process of the present invention. The examples that follow are detergent compositions that may be produced using the cleaning surfactants of the present invention.

### Example 9

### Granular Laundry Detergent

| | A | B | C | D | E |
|---|---|---|---|---|---|
| | wt % | wt % | wt % | wt % | wt % |
| C₁₁₋₁₂ Linear alkyl benzene | 13-25 | 13-25 | 13-25 | 13-25 | 9-25 |
| sulphonate | | | | | |
| C₁₂₋₁₈ Ethoxylate Sulfate | — | — | 0-3 | — | 0-1 |
| C₁₄₋₁₅ alkyl ethoxylate (EO = 7) | 0-3 | 0-3 | — | 0-5 | 0-3 |
| Dimethyl hydroxyethyl lauryl | — | — | 0-2 | 0-2 | 0-2 |
| ammonium chloride | | | | | |
| Sodium tripolyphosphate | 20-40 | — | 18-33 | 12-22 | 0-15 |
| zeolite | 0-10 | 20-40 | 0-3 | — | — |
| silicate builder | 0-10 | 0-10 | 0-10 | 0-10 | 0-10 |
| Carbonate | 0-30 | 0-30 | 0-30 | 5-25 | 0-20 |
| diethylene triamine penta | 0-1 | 0-1 | 0-1 | 0-1 | 0-1 |
| acetate | | | | | |
| polyacrylate | 0-3 | 0-3 | 0-3 | 0-3 | 0-3 |
| Carboxy Methyl Cellulose | 0.2-0.8 | 0.2-0.8 | 0.2-0.8 | 0.2-0.8 | 0.2-0.8 |
| Percarbonate | 0-10 | 0-10 | 0-10 | 0-10 | 0-10 |
| nonanoyloxybenzenesulfonate | — | — | 0-2 | 0-2 | 0-2 |
| tetraacetylethylenediamine | — | — | 0-0.6 | 0-0.6 | 0-0.6 |
| Zinc Phthalocyanine | — | — | 0-0.005 | 0-0.005 | 0-0.005 |
| Tetrasulfonate | | | | | |
| Brightener | 0.05-0.2 | 0.05-0.2 | 0.05-0.2 | 0.05-0.2 | 0.05-0.2 |
| MgSO₄ | — | — | 0-0.5 | 0-0.5 | 0-0.5 |
| ENZYMES | 0-0.5 | 0-0.5 | 0-0.5 | 0-0.5 | 0-0.5 |
| MINORS (perfume, dyes, | balance | balance | balance | balance | balance |
| suds stabilizers) | | | | | |

### Example 10

### Liquid Laundry Detergent

| | A | B | C | D | E | F⁵ |
|---|---|---|---|---|---|---|
| Ingredient | wt % | wt % | wt % | wt % | wt % | wt % |
| sodium alkyl ether sulfate | 14.4% | 14.4% | | 9.2% | 5.4% | |
| linear alkylbenzene | 4.4% | 4.4% | 12.2% | 5.7% | 1.3% | |
| sulfonic acid | | | | | | |
| alkyl ethoxylate | 2.2% | 2.2% | 8.8% | 8.1% | 3.4% | |
| amine oxide | 0.7% | 0.7% | 1.5% | | | |
| citric acid | 2.0% | 2.0% | 3.4% | 1.9% | 1.0% | 1.6% |
| fatty acid | 3.0% | 3.0% | 8.3% | | | 16.0% |
| protease | 1.0% | 1.0% | 0.7% | 1.0% | | 2.5% |
| amylase | 0.2% | 0.2% | 0.2% | | | 0.3% |
| lipase | | | | 0.2% | | |
| borax | 1.5% | 1.5% | 2.4% | 2.9% | | |
| calcium and sodium | 0.2% | 0.2% | | | | |
| formate | | | | | | |
| formic acid | | | | | | 1.1% |
| sodium polyacrylate | | | | | 0.2% | |
| sodium polyacrylate | | | | 0.6% | | |
| copolymer | | | | | | |
| DTPA¹ | 0.1% | 0.1% | | | | 0.9% |
| DTPMP² | | | 0.3% | | | |
| EDTA³ | | | | | 0.1% | |
| fluorescent whitening | 0.15% | 0.15% | 0.2% | 0.12% | 0.12% | 0.2% |
| agent | | | | | | |
| ethanol | 2.5% | 2.5% | 1.4% | 1.5% | | |
| propanediol | 6.6% | 6.6% | 4.9% | 4.0% | | 15.7% |
| sorbitol | | | | 4.0% | | |
| ethanolamine | 1.5% | 1.5% | 0.8% | 0.1% | | 11.0% |
| sodium hydroxide | 3.0% | 3.0% | 4.9% | 1.9% | 1.0% | |
| sodium cumene sulfonate | | | 2.0% | | | |
| silicone suds suppressor | | | 0.01% | | | |
| perfume | 0.3% | 0.3% | 0.7% | 0.3% | 0.4% | 0.6% |
| opacifier⁴ | | | 0.30% | 0.20% | | 0.50% |
| water | balance | balance | balance | balance | balance | balance |
| | 100.0% | 100.0% | 100.0% | 100.0% | 100.0% | 100.0% |

¹diethylenetriaminepentaacetic acid, sodium salt
²diethylenetriaminepentakismethylenephosphonic acid, sodium salt
³ethylenediaminetetraacetic acid, sodium salt
⁴Acusol OP 301

### Example 11

### Liquid Dish Handwashing Detergents

| Composition | A | B |
|---|---|---|
| C₁₂₋₁₃ Natural AE0.6S | 29.0 | 29.0 |
| C₁₀₋₁₄ mid-branched Amine Oxide | — | 6.0 |
| C₁₂₋₁₄ Linear Amine Oxide | 6.0 | — |
| SAFOL ® 23 Amine Oxide | 1.0 | 1.0 |
| C₁₁E₉ Nonionic¹ | 2.0 | 2.0 |
| Ethanol | 4.5 | 4.5 |
| Sodium cumene sulfonate | 1.6 | 1.6 |
| Polypropylene glycol 2000 | 0.8 | 0.8 |
| NaCl | 0.8 | 0.8 |
| 1,3 BAC Diamine² | 0.5 | 0.5 |
| Suds boosting polymer³ | 0.2 | 0.2 |
| Water | Balance | Balance |

¹Nonionic may be either C₁₁ Alkyl ethoxylated surfactant containing 9 ethoxy groups.
²1,3, BAC is 1,3 bis(methylamine)-cyclohexane.
³(N,N-dimethylamino)ethyl methacrylate homopolymer

### Example 12

### Automatic Dishwasher Detergent

| | A | B | C | D | E |
|---|---|---|---|---|---|
| Polymer dispersant¹ | 0.5 | 5 | 6 | 5 | 5 |
| Carbonate | 35 | 40 | 40 | 35-40 | 35-40 |
| Sodium | 0 | 6 | 10 | 0-10 | 0-10 |
| tripolyphosphate | | | | | |
| Silicate solids | 6 | 6 | 6 | 6 | 6 |
| Bleach and bleach | 4 | 4 | 4 | 4 | 4 |
| activators | | | | | |
| Enzymes | 0.3-0.6 | 0.3-0.6 | 0.3-0.6 | 0.3-0.6 | 0.3-0.6 |
| Disodium citrate | 0 | 0 | 0 | 2-20 | 0 |
| dihydrate | | | | | |
| Nonionic surfactant² | 0 | 0 | 0 | 0 | 0.8-5 |
| Water, sulfate, | Balance | Balance to | Balance | Balance | Balance |
| perfume, dyes and | to 100% | 100% | to 100% | to 100% | to 100% |
| other adjuncts | | | | | |

¹Such as ACUSOL ® 445N available from Rohm & Haas or ALCOSPERSE ® from Alco.
²such as SLF-18 POLY TERGENT from the Olin Corporation.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All document cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A process of decarboxylation of fats and oils comprising the steps of:
a. supplying a feedstock comprising natural fats, oils or mixtures thereof to a reaction vessel,
b. subjecting the feedstock to an activated acidic catalyst essentially free of Group VIII metals,
c. purging the reaction vessel with an inert gas; and
d. subjecting the feedstock and catalyst mixture to a temperature of from about 250° C. to about 500° C. and a pressure of from about 0.01 mm to about 4000 psig.

2. A process according to claim 1 wherein the process is a batch, semi-continuous or continuous process.

3. A process according to claim 2 wherein the process is a batch process.

4. A process according to claim 2 wherein the process is a continuous process.

5. A process according to claim 1 wherein the natural fat or oil is selected from the group consisting of C₈₋₂₂ fatty acids, mono- and di-glycerides of C₈₋₂₂ fatty acids, C₁₋₄ alkyl esters of C₈₋₂₂ fatty acids, triglycerides with C₈₋₂₂ fatty acid, palm oil, coconut oil, tallow fat, and mixtures thereof.

6. A process according to claim 1 wherein the feedstock and catalyst are subject to a temperature of from about 300° C. to about 400° C. and a pressure of from about 100 psig to about 500 psig.

7. A process according to claim 1 wherein the catalyst is a beta zeolite.

8. A process according to claim 1 wherein the catalyst is an acidic mordenite.

9. A process according to claim 1 wherein the catalyst is a zeolite chemically treated by a Group VI-B metal.

10. A process according to claim 1 wherein the feedstock is subjected to a multi-catalyst systems comprising two or more different activated acidic catalysts essentially free of Group VIII metals.

11. A process according to claim 1 wherein the feedstock is subjected to a multi-catalyst system comprising at least one activated acidic catalyst and from 0.001% to about 10% of the total catalyst of a Group VIII metal or Group VIII metal oxide.

12. A process for producing olefins comprising the steps of:
i) producing a paraffin by the process of decarboxylation of fats and oils according to claim 1; and
ii) converting the paraffin to any olefin by dehydrogenation.

13. A process of making a cleaning surfactant comprising the steps of:
1) producing paraffins, olefins or mixtures thereof by a process comprising the steps of:
a. supplying a feedstock comprising natural fats, oils or mixtures thereof to a reaction vessel,
b. subjecting the feedstock to an activated acidic catalyst essentially free of Group VIII metals,
c. purging the reaction vessel with an inert gas; and
d. subjecting the feedstock and catalyst mixture to a temperature of from about 250° C. to about 500° C. and a pressure of from about 0.01 mm to about 4000 psig;
2) functionalizing the paraffins, olefins or mixtures thereof by a process selected from the group consisting of OXO alcohol formation and alkylation; and
3) optionally, derivatizing the product of step 2 by a process selected from the group consisting of sulfonation and alkoxylation.

14. A detergent composition comprising cleaning surfactants made by the process of claim 13.

15. A detergent composition according to claim 14 where the detergent composition is selected from the group consisting of liquid laundry detergents, granular laundry detergents, hand dish detergents, automated dish-washer detergents, hard surface cleaners, solid soaps, liquid soaps, and hair shampoos.
